(19) United States
(12) Patent Application Publication
(10) Pub. No.: US 2023/0389843 A1
(43) Pub. Date: Dec. 7, 2023

(54) ATTENTION DEFICIT HYPERACTIVITY DISORDER DIAGNOSIS METHOD BASED ON VIRTUAL REALITY AND ARTIFICIAL INTELLIGENCE, AND SYSTEM FOR IMPLEMENTING SAME

(71) Applicant: HIPPO T&C, INC., Suwon-si (KR)

(72) Inventors: Tai-Myoung CHUNG, Suwon-si (KR); Yoo Sook JOUNG, Suwon-si (KR); Soohwan OH, Suwon-si (KR); Donggyu LEE, Suwon-si (KR); Seungho RYU, Suwon-si (KR)

(73) Assignees: HIPPO T&C, INC., Suwon-si (KR); RESEARCH & BUSINESS FOUNDATION SUNGKYUNKWAN UNIVERSITY, Suwon-si (KR); SAMSUNG LIFE PUBLIC WELFARE FOUNDATION, Seoul (KR)

(21) Appl. No.: 18/033,657

(22) PCT Filed: Oct. 27, 2020
(86) PCT No.: PCT/KR2020/014698

§ 371 Date: Apr. 25, 2023

(51) Int. Cl. A61B 5/16; A61B 5/00; A63F 13/80; G16H 50/20; G16H 10/20

(52) U.S. Cl. CPC A61B 5/168; A61B 5/7267; A63F 2300/8082; G16H 50/20; G16H 10/20; A63F 13/80

(57) ABSTRACT

The present invention relates to a attention deficit hyperactivity disorder psychological test diagnosis method based on virtual reality and artificial intelligence, and a system for implementing same, the method comprising the steps of: loading a plurality of pieces of personal questionnaire data; training AI on the basis of the personal questionnaire data; storing measurement data acquired when a person to be tested uses VR content for diagnosis; classifying attention deficit hyperactivity disorder (ADHD) classes by using the AI on the basis of the measurement data.

### TECHNICAL FIELD

The present disclosure relates to an attention deficit hyperactivity disorder psychological test diagnosis method and a system for implementing the same and, more particularly, to a method for diagnosing Attention Deficit Hyperactivity Disorder (ADHD) based on artificial intelligence to replace a ADHD test for children and adolescent and a system for implementing the method.

### BACKGROUND ART

Attention Deficit Hyperactivity Disorder (ADHD) appears in the preschool age and makes normal learning and social life impossible for children and adolescents. 70% of children diagnosed with ADHD in their childhood maintain the disease until adolescence, and 60% of the diagnosed children exhibit the symptoms even when they become adulthood.

In Korea, diagnosis and treatment of ADHD began in the 1960s, but the diagnosis and treatment have not been properly conducted until relatively recent years due to the social indifference and the lack of awareness.

Thinking that children are just immature or going through puberty, most parents do not properly recognize the symptoms of ADHD and feel burdened to visit the psychiatric department, which is the reason why the diagnosis is not made properly.

In addition, there is a problem in that the number of patients to be handled with in the psychiatric department is small because it takes a long time for testing and questionnaire, and a mental disorder may be classified differently depending on a clinician who interprets test results.

### DISCLOSURE

### Technical Problem

The present disclosure provides a method for diagnosing Attention Deficit Hyperactivity Disorder (ADHD) based on artificial intelligence to replace a ADHD test for children and adolescent, and a system for implementing the method.

### Technical Solution

In order to solve the above problem, there may be provided method for diagnosing Attention Deficit Hyperactivity Disorder (ADHD) based on virtual reality and artificial intelligence (AI) according to the present disclosure, the method including: loading a plurality of individual questionnaire data; training AI based on the questionnaire data; storing measurement data acquired when a person to be tested uses a VR content for diagnosis; and classifying a ADHD class using the AI based on the measurement data.

Meanwhile, questionnaire data may include a Korean Child Behavior Checklist (K-CBCL) result, a Continuous Performance Test (CPT) result, and the Diagnostic and Statistical Manual (DSM) for mental disorders published by the American Psychiatric Association (APA).

Meanwhile, the training of the AI further may include a class adding operation of adding a sub-class to the class classification included in the questionnaire data.

In addition, in the training of the AI, the training may be performed after assigning labels under supervised learning, and clusters are configured by class.

In addition, the training the AI may include performing unsupervised learning, classifying similar groups using a k-Nearest Neighbor (k-NN) clustering algorithm, and assigning a class to each similar group.

Meanwhile, in the training of the AI, the training may be performed after assigning labels under supervised learning, and clusters may be configured by class.

Meanwhile, the classes may include a primarily inattentive type, a primarily hyperactive type, and a combined type.

Furthermore, the classes may include sub-classes, and the sub-classes may include the person's symptoms that become severe in specific situations, and complications with other mental diseases other than ADHD.

Also, the storing of the measurement data may include data on a head direction and a gaze direction, which are acquired when the person to be tested is exposed to the VR content.

In addition, in the storing of the measurement data, data received from a gyro sensor provided in a Head Mount Display (HMD) worn on the person, an eye tracker, and a VR manipulation stick hold and manipulated by the person may be stored.

Meanwhile, the VR content may induce identifying a relationship between objects in a virtual space or induce a specific action in response to an instruction.

Meanwhile, the content may include at least one of the following: an automobile parts assembly game for sequentially moving and assembling a plurality of automobile parts arranged in a virtual space; and a baseball hitter games in which baseballs of various colors approach toward the person in the virtual space and balls of a specific color to hit is instructed.

Meanwhile, in the specifying of the ADHD symptoms, a class may be specified by digitizing an evaluation factor for each evaluation item based on the measurement data.

In addition, in the specifying of the ADHD symptoms, at least one of the following in measurement data acquired by the person playing the automobile assembly game may be digitized as the evaluation factor: a number of times following a part assembling order according to a predetermined instruction; a number of times assembling parts properly; a time taken to assemble parts; a time out of path of workflow; a time of eye gaze focused on an instructed target; a time of eye gaze distracted from an instructed target; a time of hyperactivity of a left hand without following instructions; a time of hyperactivity of a right hand without following instructions; and a number of times a content is retried and a number of abandonment of a content.

In addition, in the specifying of the ADHD symptoms, at least one of the following in measurement data acquired by the person performing the baseball hitter game is digitized as the evaluation factor: a number of times hitting a baseball of an instructed color; a number of times hitting a baseball correctly; a time taken to complete an entire content; a time of eye gaze focused on an instructed target; a time of eye gaze distracted from an instructed target; a time of hyperactivity of a left hand without following instructions; a time of hyperactivity of a right hand without following instructions; and a number of abandonment of a content.

Meanwhile, the method may further include: after the specifying of the ADHD symptom, suggesting a treatment plan for the ADHD symptom.

In addition, there may be provided a system for diagnosing Attention Deficit Hyperactivity Disorder (ADHD) based on virtual reality and an artificial intelligence according to the present disclosure, the system including: a data collecting module comprising personal questionnaire data interviewed in advance; an AI module configured to perform learning based on the questionnaire data collected from the data collecting module and categorize and generate evaluation items; a VR module to be worn and gripped by a person to be tested; a content module comprising a content to be implemented in the VR module; a measurement data storage module configured to store measurement data acquired from the VR module as the person uses the content; a class classifying module configured to evaluate each evaluation item based on the measurement data and determine an ADHD class; a treatment plan recommending module configured to suggest a treatment plan according to the classified ADHD class.

Meanwhile, the content module may include a content that induces identifying a relationship between objects in a virtual space or induces a specific action in response to an instruction.

Meanwhile, the content may include: an automobile parts assembly game for sequentially moving and assembling a plurality of automobile parts arranged in a virtual space; and a baseball hitter games in which baseballs of various colors approach toward the person in the virtual space and balls of a specific color to hit is instructed.

Meanwhile, the class classifying module may specify the class by digitizing an evaluation factor for each evaluation item based on the measurement data.

### ADVANTAGEOUS EFFECTS

The method for diagnosing Attention Deficit Hyperactivity Disorder (ADHD) based on virtual reality and artificial intelligence according to the present disclosure and a system implementing the same enable a user and/or a doctor to quickly and accurately identify concentration deficit hyperactivity disorder based on the results of this system, It has the effect of being able to simply receive the appropriate treatment plan.

### DESCRIPTION OF DRAWINGS

FIG. 1 is a conceptual diagram illustrating the configuration of a system for diagnosing Attention Deficit Hyperactivity Disorder (ADHD) based on virtual reality and artificial intelligence (AI) according to an embodiment of the present disclosure.

FIG. 2 is a flow chart illustrating a method for diagnosing Attention Deficit Hyperactivity Disorder (ADHD) based on virtual reality and artificial intelligence (AI) according to another embodiment of the present disclosure.

FIG. 3 is a detailed flowchart illustrating an AI training operation.

FIG. 4 is an example of VR content implemented to obtain measurement data.

FIG. 5 is another example of VR content implemented to obtain measurement data.

FIG. 6 is a conceptual diagram showing data and evaluation items, which are processed when diagnosing a class based on measurement data.

FIG. 7 is a conceptual diagram of an AI training operation in class classification.

FIG. 8 is a conceptual diagram of an operation of proposing a treatment plan according to a class.

### MODE FOR DISCLOSURE

Hereinafter, a method for diagnosing Attention Deficit Hyperactivity Disorder (ADHD) based on virtual reality and artificial intelligence according to an embodiment of the present disclosure and a system implementing the same will be described in detail with reference to the accompanying drawings. In addition, in description of the following embodiments, the names of components used in the following description may be referred to as other names in this field. However, even if modified embodiments are selected, they may be considered as equivalent configurations, as long as there are functional similarity and identity. Further, the reference numerals of the components are provided for the convenience of description. However, those indicated by the reference numerals in the drawings do not limit the components to the range shown in the drawings. Similarly, even if embodiments obtained by modifying some of the configurations in the drawings are selected, they may be considered as equivalent configurations, as long as there are functional similarity and identity. Further, when components are recognized as components that should be included at the level of those skilled in the art, they are not described.

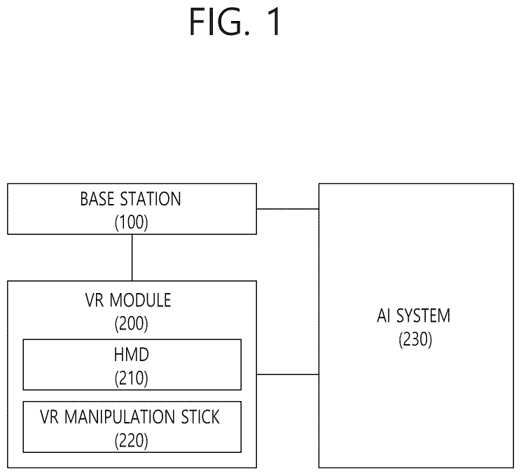

Hereinafter, a system for diagnosing Attention Deficit Hyperactivity Disorder (ADHD) based on virtual reality and artificial intelligence (AI) according to the present disclosure will be described with reference to FIG. 1.

FIG. 1 is a conceptual diagram illustrating the configuration of a system for diagnosing ADHD based on virtual reality and artificial intelligence (AI) according to an embodiment of the present disclosure.

Referring to FIG. 1, a system for diagnosing ADHD based on virtual reality and artificial intelligence according to the present disclosure may include a base station 100, a VR module 200, and an AI system.

The base station 100 may include a data collecting module for collecting and storing questionnaire data. Since the data collecting module may be configured as a widely used configuration including a conventional storage device, a further detailed description thereof will be omitted.

The questionnaire data may include data of medical questionnaire of a patient to be tested, as a conventional method in a related art.

Questionnaire data may include at least one questionnaire of a Korean Child Behavior Checklist (K-CBCL) result, a Continuous Performance Test (CPT) result, and the Diagnostic and Statistical Manual (DSM) for mental disorders published by the American Psychiatric Association (APA). Preferably, the questionnaire may include all of the medical questionnaires described above. For example, the diagnostic and statistical manual of mental disorders-

issued by the American Psychiatric Association (APA) may include items as shown in Table 1 below as criteria for determining ADHD.

TABLE 1
Failing to give close attention to details (1a)
Making careless mistakes (1a)
Not listening when others are talking (1c)
Inability to pay sustained attention (1b) - the subject should not be very
interesting/computer games or hobbies)
Failing to finish tasks or tasks (1d)
Avoiding, disliking, or reluctant to engage in tasks that require sustained
mental effort (strain) (1f)
Easily distracted by extraneous stimuli (1h)
Having difficulty in concentrating when a task is changed (Clinical)
Having difficulty completing tasks which are preoccupied (Clinical)
Difficulty performing two tasks at the same time (clinical)
Difficulty organizing tasks and activities (1e)
Being forgetful in daily activities (1i)
Often losing things necessary for tasks or activities (1g)
Fidgeting with hands and feet, hard to keep the hands and feet still,
or wriggling even when seated (2a)
Leaving seat in classroom or other situations in which
remaining seated is required (2b)
Excessively running around or climbing inappropriately (2c)
Inability to engage in leisure activities or play quietly (2d)
Being “active without rest” or acting like “unwound automobile”(2e)
Talking too much (2f)
Talking in an excessively loud voice (HL1)
Cannot wait for one's turn (2h)
Interfering with or infringing on the activities of others (2i)
Sudden emotional response (Barkley)
Having difficulty in controlling anger easily (Barkley)
Rushed to answer questions before they are finished (2g)
Interfering with or infringing on the activities of others (21)

The VR module 200 is configured to measure a behavior of a subject in a specific situation. The VR module 200 may include a Head Mound Display (HMD) 210 and a VR manipulation stick 220.

Although not shown, the VR module 200 may include a speaker, a display part, a gyro sensor, and an eye tracker. The VR module 200 is configured to expose virtual reality in a specific situation to the subject and to use content. In this case, the VR module 200 may transmit measurement data by sensing motions of the subject by the gyro sensor, the eye tracker, and the VR manipulation stick 220. Meanwhile, although not shown, a microphone may be additionally included to acquire voice data of the subject.

For example, it is configured to acquire data on behaviors such as the subject's changing a looking direction by turning his or her head, the subject's walking or running, the subject's changing a direction of his or gaze, or the subject's moving his or her hand.

The AI system generates evaluation criteria by pre-processing the questionnaire data, and classifying as an ADHD class based on the evaluation criteria by using measurement data acquired while the subject uses VR content.

In addition, the AI system may be configured to suggest an appropriate treatment plan. The AI system may include a widely used processor, and may load questionnaire data and perform learning in conjunction with the base station 100.

Meanwhile, the AI system may include a measurement data storage module, a class classifying module, and a treatment plan recommending module, the modules classified by functions, and may include algorithms that perform the respective functions.

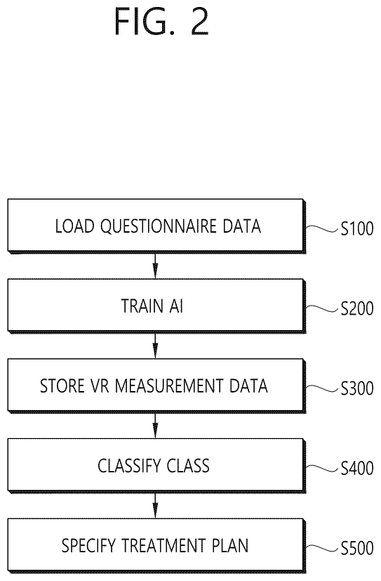

Hereinafter, a method for diagnosing ADHD based on virtual reality and artificial intelligence according to another embodiment of the present disclosure will be described in detail with reference to FIGS. 2 to 8.

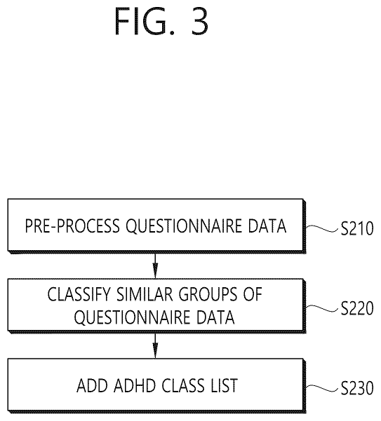

FIG. 2 is a flowchart of a method for diagnosing ADHD based on virtual reality and artificial intelligence another embodiment according to the present disclosure, and FIG. 3 is a detailed flowchart of an AI training operation.

Referring to FIG. 2, the method for diagnosing ADHD based on virtual reality and artificial intelligence according to the present disclosure may include loading questionnaire data, training AI, storing VR measurement data, classifying a class, and specifying a treatment plan.

The operation S100 of loading questionnaire data corresponds to an operation of loading data for a conventional medical questionnaire for AI training. As mentioned above, a plurality of individual questionnaire data may include at least one questionnaire of Korean Child Behavior Checklist (K-CBCL) evaluation results, Continuous Performance Test (CPT) results, and the Diagnostic and Statistical Manual (DSM) for mental disorders published by the American Psychiatric Association (APA).

The operation S200 of training AI corresponds to an operation of categorizing the questionnaire data by training the AI and classifying an ADHD class. The operation of training AI may be performed through unsupervised learning or supervised learning.

When the operation of training AI is performed through unsupervised learning, a k-Nearest Neighbor (k-NN) clustering algorithm may be used to classify the data into similar groups, and a class may be assigned to a similar group. On the other hand, when the operation of training AI is performed through supervised learning, AI may be trained after label assignment under the guidance of an expert, and clusters may be configured by class.

Describing the operation S200 of training AI with reference to FIG. 3, the operation S200 of training AI may be include pre-processing the questionnaire data in operation S210, classifying similar groups of the questionnaire data in operation S220, and adding an ADHD class list in operation S230.

The operation S210 of pre-processing the questionnaire data corresponds to an operation of converting the data for diagnosing ADHD into an appropriate form for AI to learn.

The operation S220 of classifying the similar groups of the questionnaire data is configured to categorize the evaluation items. For example, inattention, hyperactivity, and impulsivity may be included as major categories of the evaluation items of Table 1 described above. The inattention includes general inattention, executive function, and working memory as subcategories. The hyperactivity and impulsivity may include behavior and language as subcategories.

For example, clinical observation items to be categorized may include Table 2 below.

TABLE 2

Making mistakes by not reading questions thoroughly
Not reading questions thoroughly and leaving them unsolved
Taking too much time to do a lot of details
Making careless mistakes in school homework
Leaving the back page of the test paper unsolved
Not reviewing answers to homework questions
Not understanding what parents/teachers say
Being hazy or dazed
Paying attention only when making an eye contact or
when someone raises his or her voice
Often need to talk back
In need to repeat the same question
Easy to be distracted
In need of a lot of structure and steps to not get distracted

In addition, in the operation S220, tokenization is performed according to similar group classification, and tokens are classified into a token for a fixed constant such as a place and a token for a variable that represents a patient's action, reaction, or the like. The classified tokens are used to digitize measurement data later, and eventually used as the basis for ADHD class classification.

The operation S230 of adding an ADHD class corresponds to adding an additional sub-class distinguished from conventional ADHD classes. The ADHD classes are based on the conventional classes of a primarily inattentive type, a primarily hyperactive type, and a combined type. As the sub-class added in this operation, a class for distinguishing a symptom that becomes severe in a specific situation is added. For example, in this operation, “being inattentive when instructions are given” and “being unconditionally hyperactive in response to a teacher's instructions” may be added as sub-classes. In addition, in this operation, complications with other mental disorders other than ADHD may be added as a subclass. For example, combinations between ADHD and depression, between ADHD and anxiety disorder, and between ADHD and tic disorder may be added as sub-classes for complications with other mental disorders.

Referring back to FIG. 2, the operation S300 of storing VR measurement data corresponds to acquiring data that is acquired when a person to be tested uses a VR content. The operation of storing the VR measurement data may be configured to expose a user to a VR environment, give a predetermined instruction in a predetermined environment, and measure the user's behavior in response to the instruction. Eye tracking data, head tracking data, and voice data obtained from the HMD may be measured according to the person's behavior, and movement of a hand with a VR control stick may be measured.

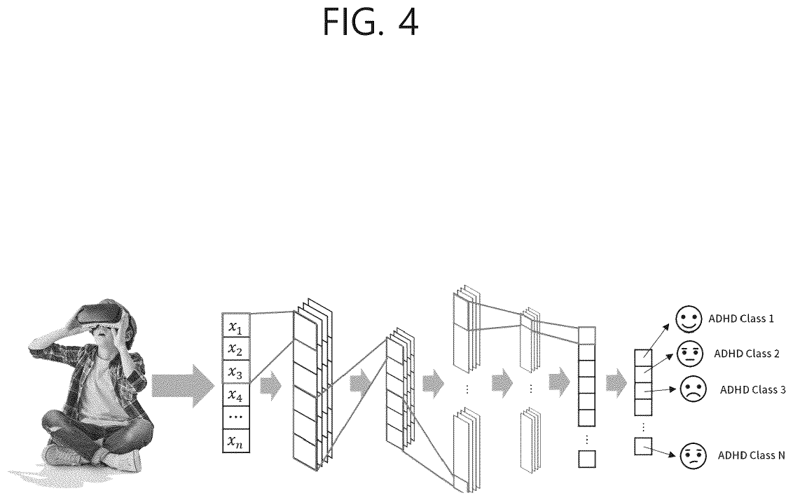

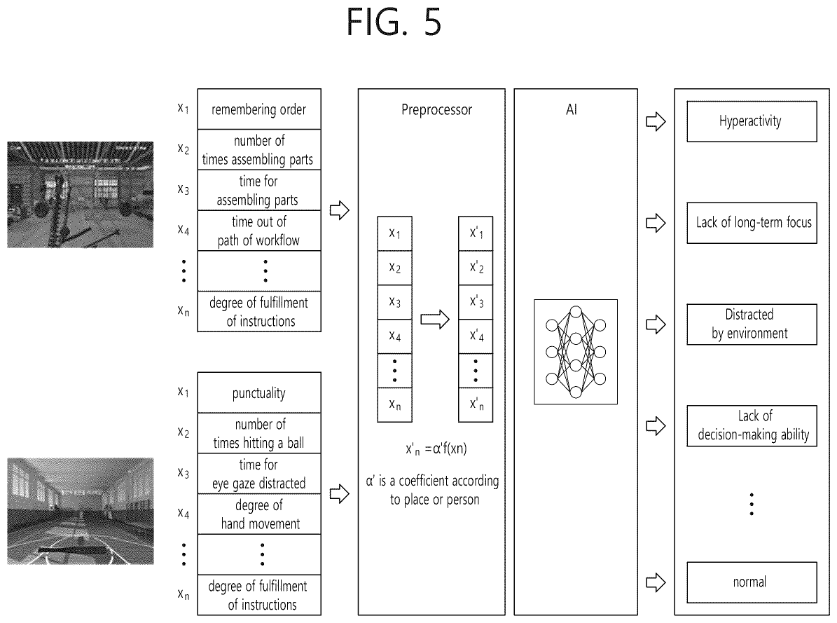

FIG. 4 is an example of a VR content implemented to acquire measurement data, and FIG. 5 is another example of a VR content implemented to acquire measurement data.

Referring to FIGS. 4 and 5, a VR content may include virtual reality data used for a person to be tested to recognize an instruction and induce a specific action in a predetermined situation, such as assessment of attention when identifying a relationships between objects or recognizing a relationship between multiple objects.

As an example, the VR content may include an automobile assembly game, a baseball hitter game, and the like. In the case of the automobile assembly game, a task to move parts located in different positions to the car within a designated area according to an order indicated may be given. In addition, the baseball hitter game may be a game in which balls of various colors comes toward a person to be tested from a far side of the screen and only balls of instructed color must be hit.

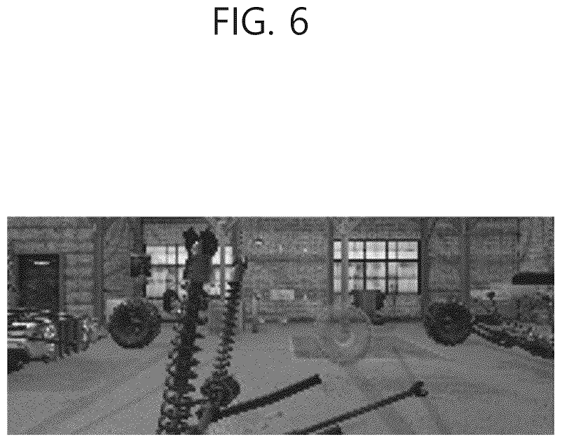

FIG. 6 is a conceptual diagram showing data and evaluation items, which are processed when diagnosing a class based on measurement data.

Referring to FIG. 6, the operation S400 of classifying a class corresponds to classifying an ADHD class based on measurement data of a person to be tested according to a VR content and an instruction. The operation of classifying a class may be performed in the aforementioned AI system, and the AI classifies the class using a result of matching the obtained measurement data with tokenized evaluation items.

Meanwhile, the measurement data may be interpreted to have different meanings depending on the content to be implemented. For example, observation items interpreted according to the measured data may include items shown in Table 3 below. Therefore, in this operation, measurement data is analyzed in association with the content and classes are classified.

TABLE 3
in response to very easy questions, going through the questions very
quickly and but making incorrect answer
Skipping provided questions without answering
Frequently exceeding the time limit when performing complex, time-
limited tasks.
Skipping provided questions without answering
Failing to respond to requests to review answers to questions
provided
Increased rate of incorrect answers to questions provided in the form
of voice
Distracted gaze while audio questions or tasks are being provided.
Or failure of moving the gaze along with a moving target.
Increased rate of correct answers when audio questions are provided
with a louder-than-average voice, heightened intonation, quick screen
transitions, or movement of the questions.
Task performance increased significantly in the presence of voice
feedback.
Frequently selecting items to listen to questions again.

A different evaluation factor for each observation item may be selected for each content.

For example, in the case of a car assembly game, specific evaluation factors according to observation items may include Table 4 below.

TABLE 4

Number of times remembering the order of assembling parts
(Correctness, times)
Number of times parts are assembled (Correctness)
Time for assembling parts (seconds)
Time out of path of workflow (seconds)
Time of eyes focused on instructions (eye-tracking, seconds)
Time of eyes distracted (eye-tracking, seconds)
Time of abnormal movement of left hand (seconds)
Time of abnormal movement of right hand (seconds)
Number of times a content is retried (times)
Number of times a content is abandoned (times)

In addition, as another example, in the case of the baseball hitter game, specific evaluation factors according to observation items may include Table 5 below.

TABLE 5

Number of times remembering baseball colors (Correctness, times)
Number of times hitting baseballs (Correctness)
Total execution time (seconds)
Time of eyes focused on instructions (eye-tracking, seconds)
Time of eyes distracted (eye-tracking, seconds)
Time of abnormal movement of left hand (seconds)
Time of abnormal movement of right hand (seconds)
Number of times a content is abandoned (times)

Meanwhile, if the content is an automobile assembly game, remembering an order, the number of times assembling parts, the time for assembling parts, the time out of path of workflow, and a degree of fulfillment of instructions are analyzed to pre-process measurement data for each evaluation item. In addition, based on the analyzed data, pre-processing is performed as follows.

X_{garage,p}_{₁}={(x₁, 5), (x₂, 7), x₃, 12)(x₄, 5)}

X̅_{garage,p}_{₁}={(x′₁, 0.3), (x′₂, 0.5), (x′₃, 0.9)(x′₄0.3)}

After the pre-processing, the AI system performs class classification based on pre-processed and digitized data. Class classification may be determined as follows.

$${Y}_{garage , {p}_{1}} = f ( \overline{x} ) = \{ Hyperactivity , Distractions \}$$

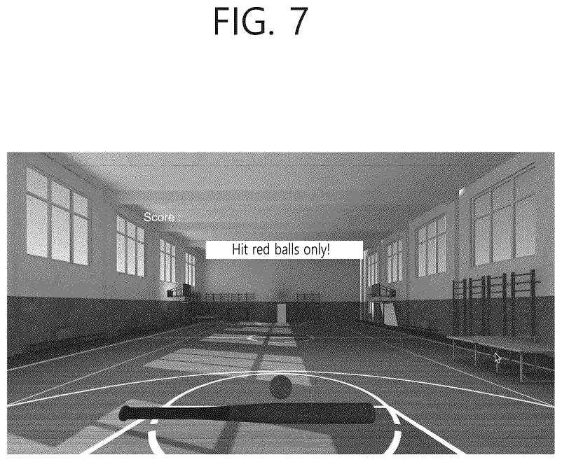

FIG. 7 is a conceptual diagram of an AI training operation in class classification.

Referring to FIG. 7, upon class classification, the AI system pre-processes measurement data based on the measurement data, and then performs learning using an online learning technique for real-time update of learning results.

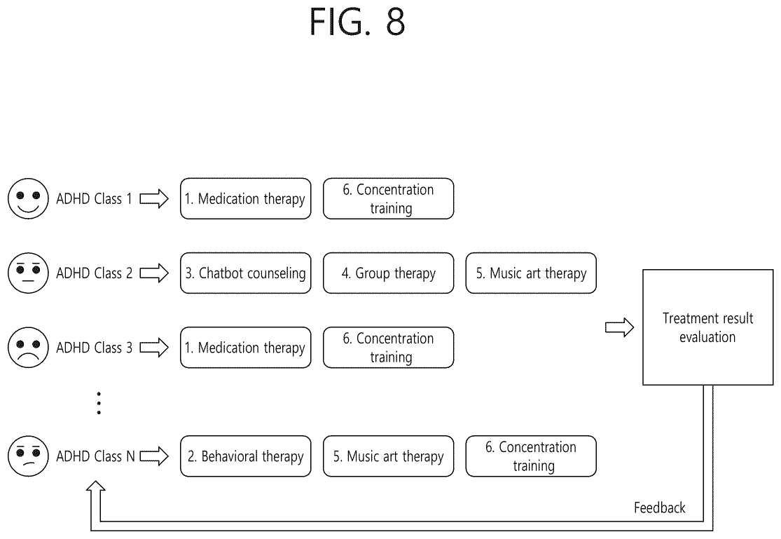

FIG. 8 is a conceptual diagram of an operation of proposing a treatment plan according to a class.

The operation S500 of specifying a treatment plan corresponds to suggesting an appropriate treatment plan for a user according to a class classified by the AI. The specification of a treatment plan may be suggested according to the ADHD class, and it may be selected and suggested from a list of treatment plans currently being performed, such as medication therapy, concentration training, chatbot counseling, group therapy, music art therapy, and behavioral therapy.

Meanwhile, individual measurement data, an ADHD class, and data on the proposed treatment plan may be stored additionally for a person to be tested, and a treatment result is evaluated based on the stored data and fed back to the AI system in the operation of classifying an ADHD class and the operation of suggesting a treatment plan to thereby enhance a diagnosis algorithm, thereby improving a diagnosis rate and treatment rate of class classification.

As described above, in the method for diagnosing ADHD based on virtual reality and artificial intelligence according to the present disclosure and the system for implementing the same, it is possible to allow a user and/or doctor to quickly and accurately determine ADHD based on a result of this system and to simply provide an appropriate treatment plan.

1. A method for diagnosing Attention Deficit Hyperactivity Disorder (ADHD) based on virtual reality and artificial intelligence (AI), the method comprising:
loading a plurality of individual questionnaire data;
training AI based on the questionnaire data;
storing measurement data acquired when a person to be tested uses a VR content for diagnosis; and
classifying a ADHD class using the AI based on the measurement data.

2. The method of claim 1, wherein the questionnaire data comprises a Korean Child Behavior Checklist (K-CBCL) result, a Continuous Performance Test (CPT) result, and the Diagnostic and Statistical Manual (DSM) for mental disorders published by the American Psychiatric Association (APA).

3. The method of claim 2, wherein the training of the AI further comprises a class adding operation of adding a sub-class to a class classification included in the questionnaire data.

4. The method of claim 3, wherein in the training of the AI, an ADHD class is classified based on the questionnaire data, and evaluation items for inattention, hyperactivity, and impulsivity are generated.

5. The method of claim 4, wherein the training of the AI comprises performing unsupervised learning, classifying into similar groups using a k-Nearest Neighbor (k-NN) clustering algorithm, and assigning a class to each similar group.

6. The method of claim 4, wherein in the training of the AI, the training is performed after assigning labels under supervised learning, and clusters are configured by class.

7. The method of claim 4, wherein the classes comprises a primarily inattentive type, a primarily hyperactive type, and a combined type.

8. The method of claim 7,
wherein the classes comprises sub-classes, and
wherein the sub-classes comprise the person's symptoms that become severe in specific situations, and complications with other mental diseases other than ADHD.

9. The method of claim 4, wherein the storing of the measurement data comprises data on a head direction and a gaze direction, which are acquired when the person to be tested is exposed to the VR content.

10. The method of claim 9, wherein in the storing of the measurement data, data received from a gyro sensor provided in a Head Mount Display (HMD) worn on the person, an eye tracker, and a VR manipulation stick hold and manipulated by the person is stored.

11. The method of claim 7, wherein the VR content induces identifying a relationship between objects in a virtual space or induces a specific action in response to an instruction.

12. The method of claim 11, wherein the content comprises at least one of the following:
an automobile parts assembly game for sequentially moving and assembling a plurality of automobile parts arranged in a virtual space; and
a baseball hitter games in which baseballs of various colors approach toward the person in the virtual space and balls of a specific color to hit is instructed.

13. The method of claim 12, wherein in the specifying of the ADHD symptoms, a class is specified by digitizing an evaluation factor for each evaluation item based on the measurement data.

14. The method of claim 13, wherein in the specifying of the ADHD symptoms, at least one of the following in measurement data acquired by the person playing the automobile assembly game is digitized as the evaluation factor:
a number of times following a part assembling order according to a predetermined instruction;
a number of times assembling parts properly;
a time taken to assemble parts;
a time out of path of workflow;
a time of eye gaze focused on an instructed target;
a time of eye gaze distracted from an instructed target;
a time of hyperactivity of a left hand without following instructions;
a time of hyperactivity of a right hand without following instructions; and
a number of times a content is retried and a number of abandonment of a content.

15. The method of claim 13, wherein in the specifying of the ADHD symptoms, at least one of the following in measurement data acquired by the person performing the baseball hitter game is digitized as the evaluation factor:
a number of times hitting a baseball of an instructed color;
a number of times hitting a baseball correctly;
a time taken to complete an entire content;
a time of eye gaze focused on an instructed target;
a time of eye gaze distracted from an instructed target;
a time of hyperactivity of a left hand without following instructions;
a time of hyperactivity of a right hand without following instructions; and
a number of abandonment of a content.

16. The method of claim 13, further comprising:
after the specifying of the ADHD symptom, suggesting a treatment plan for the ADHD symptom.

17. A system for diagnosing Attention Deficit Hyperactivity Disorder (ADHD) based on virtual reality and an artificial intelligence, the system comprising:
a data collecting module comprising personal questionnaire data interviewed in advance;
an AI module configured to perform learning based on the questionnaire data collected from the data collecting module and categorize and generate evaluation items;
a VR module to be worn and gripped by a person to be tested;
a content module comprising a content to be implemented in the VR module;
a measurement data storage module configured to store measurement data acquired from the VR module as the person uses the content;
a class classifying module configured to evaluate each evaluation item based on the measurement data and determine an ADHD class;
a treatment plan recommending module configured to suggest a treatment plan according to the classified ADHD class.

18. The system of claim 17,
wherein the content module comprises a content that induces identifying a relationship between objects in a virtual space or induces a specific action in response to an instruction.

19. The system of claim 18, wherein the content comprises:
an automobile parts assembly game for sequentially moving and assembling a plurality of automobile parts arranged in a virtual space; and
a baseball hitter games in which baseballs of various colors approach toward the person in the virtual space and balls of a specific color to hit is instructed.

20. The system of claim 19, wherein the class classifying module specifies the class by digitizing an evaluation factor for each evaluation item based on the measurement data.
